(19) United States
(12) Patent Application Publication
(10) Pub. No.: US 2008/0108049 A1
(43) Pub. Date: May 8, 2008

(54) CHIMERIC PROTEIN AND ITS USE IN ELECTRON TRANSFER METHODS

(75) Inventors: Gianfranco Gilardi, London (GB); Anthony E.G. Cass, London (GB)

Correspondence Address: SUGHRUE MION, PLLC, 2100 PENNSYLVANIA AVENUE, N.W., SUITE 800, WASHINGTON, DC, 20037, US

(73) Assignee: NANOBIODESIGN LIMITED, Kent (GB)

(21) Appl. No.: 11/862,954

(22) Filed: Sep. 27, 2007

Related U.S. Application Data
(62) Division of application No. 10/485,621, filed on Aug. 30, 2004.

(30) Foreign Application Priority Data
Aug. 3, 2001 (GB) 0119042.0

(51) Int. Cl. C12N 13/00; C12Q 1/00

(52) U.S. Cl. 435/4; 435/173.2

(57) ABSTRACT

A chimeric protein comprises a redox catalytic domain from one source and an electron transfer domain from a different source. The protein is used in a method in which a substrate for the redox catalytic domain is acted on, electrons are transferred between the redox catalytic domain and the electron transfer domain and between the electron transfer domain and an electrode. The flow of current or potential at the electrode may be monitored to determine the presence or amount of a substrate which is an analyte of interest. Alternatively current may be driven through the electrode to drive reaction of the substrate, for instance to detoxify samples. The redox catalytic domain is suitably derived from a cytochrome P450, and the electron transfer domain may be flavodoxin.

This is a divisional of application Ser. No. 10/485,621, filed Aug. 30, 2004, which was a National Stage entry of PCT/GB02/03596, filed Aug. 5, 2002, which claims priority to GB 0119042.0, filed Aug. 3, 2001. The entire disclosures of the 10/485,621, PCT/GB02/03596 and GB 0119042.0 prior applications are incorporated herein by reference.

A Sequence Listing is included herein, by incorporation, via the attached Request to Use Computer-Readable Form of Sequence Listing Submitted in a Co-pending patent application in Accordance with 37 C.F.R. § 1.821(e).

### BACKGROUND OF THE INVENTION

The present invention relates to a method of carrying out an electrochemical process involving a chimeric protein and a kit.

Cytochromes P450 (P450) are highly relevant to the bio-analytical area (Sadeghi et al, 2001). They form a large family of enzymes present in all tissues important to the metabolism of most of the drugs used today, playing an important role in the drug development and discovery process (Poulos, 1995, Guengerich, 1999). They catalyse the insertion of one of the two atoms of an oxygen molecule into a variety of substrates (R) with quite broad regioselectivity, resulting in the concomitant reduction of the other oxygen atom to water, according to the reaction:
RH+O₂+2e⁻+2H⁺→ROH+H₂O

Despite their importance, applications in the bio-analytical area are difficult due to problems related to their poor interaction with electrode surfaces and the association to biological membranes of the mammalian P450. Nevertheless, an exciting potential application of these enzymes relies on the creation of electrode arrays for high-through-put screening for propensity to metabolic conversion or toxicity of novel potential drugs.

Cytochrome P450 BM3 is a soluble, catalytically self-sufficient fatty acid monoxygenase isolated from Bacillus megaterium (Narhi and Fulco, 1986 and 1987). It is particularly interesting in that it has a multi-domain structure, composed of three domains: one FAD, one FMN and one haem domain, fused on the same 119 kDA polypetidic chain of 1048 residues. Furthermore, despite its bacterial origin, P450 BM3 has been classified as a class II P450 enzyme, typical of microsomal eukaryotic P450s (Ravichandran et al., 1993); it shares 30% sequence identity with microsomal fatty acid w-hydroxylase, 35% sequence identity with microsomal NADPH:P450 reductase, and only 20% homology with other bacterial P450s (Ravichandran et al., 1993). These characteristics have suggested the use of P450 BM3 as a surrogate for mammalian P450s and this has been recently substantiated when the structure of rabbit P450 2C5 was solved (Williams et al., 2000).

Sadeghi et al., 2000a describe a chimeric protein comprising a redox catalytic domain derived from BM3 of Bacillus megaterium and flavodoxin from Desulfovibrio vulgaris [Hildenborough], expressed in the pT7 expression system. Electron transfers between the redox catalytic domain derived from BM3 and the electron transfer domain of FLD was observed by photoreducing FLD to its semiquinone form in the presence of arachidonate (substrate) bound to the redox catalytic domain of BM3 by monitoring at 450 nm under a carbon monoxide atmosphere.

### BRIEF SUMMARY OF THE INVENTION

There is provided in the invention a new method in which a chimeric protein comprising a redox catalytic domain derived from a first source and an electron transfer domain derived from a second source different to the first source is contacted with a substrate for the catalytic domain, and with an electrode, whereby the substrate is acted on by the catalytic domain, to form a product and electrons are transferred directly between the electrode and the electron transfer domain and between the electron transfer domain and the catalytic domain.

In the method, the first source and the second source differ by the genus, or the species from which they are derived, or they may be derived from the same species as one another but from different organelles or compartments in the same species. Preferably they are derived from different species.

### BRIEF DESCRIPTION OF THE DRAWINGS

FIG. 1 shows the invention applied to P450 BM3 (A) to generate a P450 catalytic domain electrochemically accessible through the fusion with the electron transfer protein flavodoxin; (B) to generate libraries of P450 BM3 enzymes with different catalytic domains to be used for pharmacological and biosensing applications.

FIG. 2 shows (A) reduction of arachidonate-bound BMP (BMP-S) by flavodoxin semiquinone (FLD_{sq}) followed at 450 nm by stopped flow spectrophotometry in the presence of carbon monoxide. (B) Plot of the limiting pseudo-first-order rate constants (kₗᵢₘ) versus the square root of the ionic strength (I) for the reaction between FLD_{sq} and BMP-S.

FIG. 3 shows cyclic voltammograms of BMP-FLD fusion protein in the absence (1, thin line) and presence (2, thick line) of neomycin on a glassy carbon electrode. The addition of carbon monoxide shifts the peak to higher potentials (3, dotted line). Potentials are reported versus saturated calomel electrode.

FIG. 4 shows a molecular biological approach to fuse the genes of BMP and FLD to generate the BMP-FLD chimera. The Nla III restriction sites were introduced by oligonucleotide directed mutagenesis.

### DETAILED DESCRIPTION

Preferably the redox catalytic domain is a haem-containing domain, preferably derived from a P450 enzyme. Preferably the haem-containing domain is a monooxygenase domain.

Preferably the electron transfer domain is a haem reductase domain and the electrode is a cathode. Preferably the electron transfer domain is a flavoprotein, such as flavodoxin from D. vulgaris or an active electron-transferring mutant form thereof.

Preferably electrons are directly transferred from the electrode to the electron transfer domain, although in some embodiments it may be possible for the electrons to be transferred via an additional electron transfer module, such as ubiquinone, or a cytochrome.

The chimeric protein preferably additionally comprises a docking sequence having a docking site for the electron transfer domain. The docking sequence may be derived from the same source as the redox catalytic domain, preferably being the docking site from the Bacillus megaterium protein BM3.

The source of the redox domain is preferably an oxygenase enzyme, such as a cytochrome P450, which is generally a monooxygenase enzyme. In one embodiment the redox catalytic domain is derived from a bacterial cytochrome P450 enzyme, most preferably from a self-sufficient enzyme such as BM3 of Bacillus megaterium. The redox catalytic domain may itself comprise components derived from multiple sources. Thus the domain may comprise a docking site for the electron transfer domain derived from one source and a substrate binding site derived from another source, such as from a different species or even genus. One source may be mammalian such as a mammalian P450 enzyme.

In the method the flow of electrons from the electrode may be measured, for instance using a current or voltage detector. It is generally desired to measure the current.

The method may be used to determine the presence or concentration, or alternatively the catabolism of an analyte of interest. In such embodiments the substrate is an analyte of interest and in the method the measurement of the flow electrons is used to detect the presence or amount of substrate.

Although it may be possible for the method to be used for methods on which electrons flow from the electrode to the electron transfer domain, it is preferable that electrons are driven from the electrode, and that the substrate is consumed. In preferred embodiments, the product is separated from the chimeric protein and, usually, recovered. In some circumstances the method is useful to detoxify a substrate, and the product may be merely disposed of without being recovered. The invention may be of use to determine the reaction of substrates, such as drugs or other compounds which may be administered or ingested by humans or other animals, with the redox domain.

In other methods the process may be used to produce products of use as commercial products. In such methods the chimeric protein may be used for repeated cycles of reaction, for instance by immobilising the protein on the electrode and recovering the product from solution. For instance the invention may be used in an electrochemical synthesis, in which current is driven through the electrode, starting material (substrate) is consumed and the desired product is synthesised and recovered from solution.

The invention also comprises a kit comprising the chimeric protein and an electrode. The electrode is generally provided in a vessel for containing an aqueous reaction medium containing the protein, and usually the substrate. The kit should have the preferred features as in the method as described above.

Immobilisation of the protein on the electrode may be by adsorption, for instance involving ionic bonding, optionally using a soluble charged species, which is able to bond counterionically to both protein and the electrode surface. Preferably immobilisation is by a covalent bond from a side chain of an amino acid residue of the electron transfer domain to the electrode surface. Methods known in the prior art for bonding proteins to surfaces, especially conductive surfaces, such as are useful for forming electrodes, may be used. For instance thiol groups of cysteine residues may be used to bond covalently to gold surfaces. (Bagby et al., 1991).

In some embodiments the kit may be provided with the chimeric protein in immobilised form. In other embodiments the chimeric protein is in water soluble form in the kit. Kits in which the protein is water soluble as supplied may include immobilising means for in situ immobilisation of the protein, for instance, comprising a multi-valent charged compound, especially neomycin.

In the invention there is also provided apparatus comprising

i) a reaction vessel containing
a) an electrode,
b) a liquid comprising in solution a substrate for the redox enzyme, and
c) the chimeric protein and

ii) a current collector electrically connected to the electrode.

The apparatus may be connected to conventional current and/or voltage monitoring means for detecting a flow of current through the current collector and the electrode and/or the potential of the electrode.

The invention is illustrated in the accompanying drawings in which:

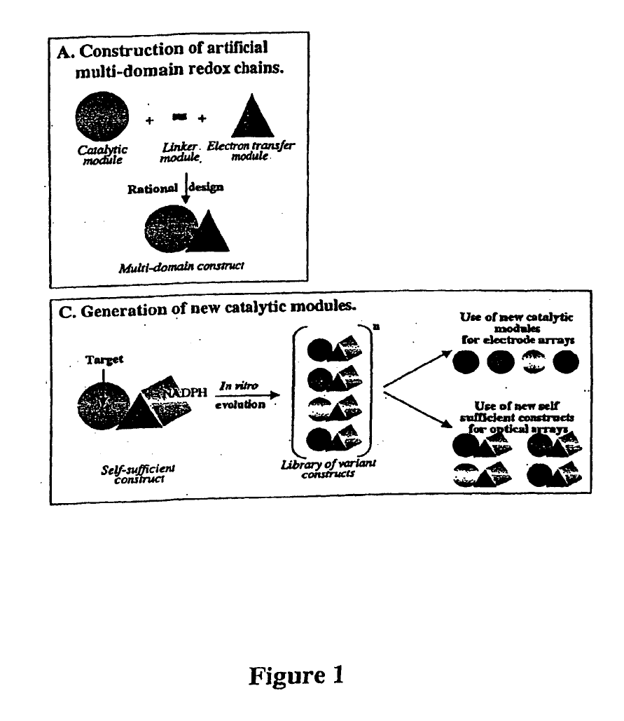

FIG. 1 shows the invention applied to P450 BM3 (A) to generate a P450 catalytic domain electrochemically accessible through the fusion with the electron transfer protein flavodoxin; (B) to generate libraries of P450 BM3 enzymes with different catalytic domains to be used for pharmacological and biosensing applications.

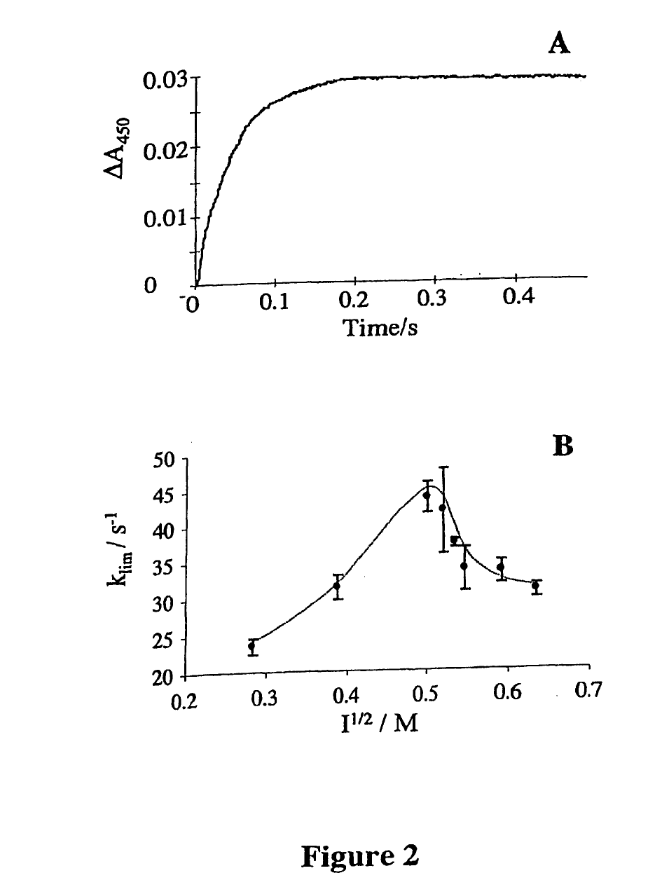

FIG. 2 shows (A) Reduction of arachidonate-bound BMP (BMP-S) by flavodoxin semiquinone (FLD_{sq}) followed at 450 nm by stopped flow spectrophotometry in the presence of carbon monoxide. (B) Plot of the limiting pseudo-first-order rate constants (kₗᵢₘ) versus the square root of the ionic strength (I) for the reaction between FLD_{sq} and BMP-S.

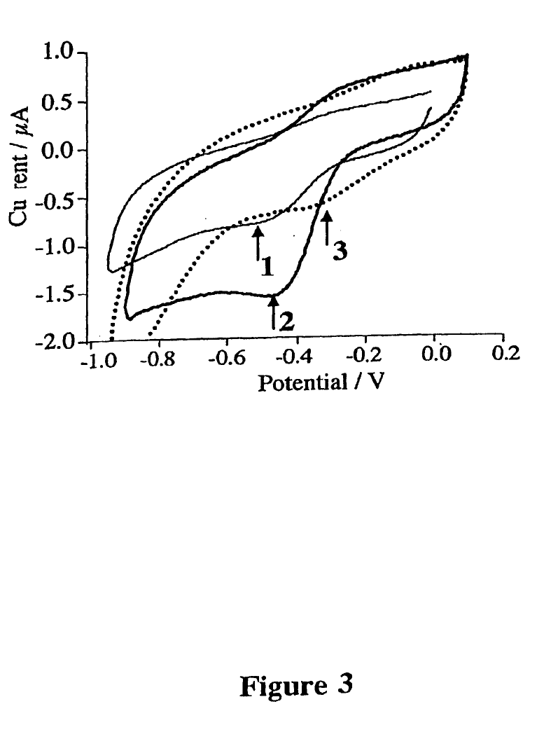

FIG. 3 shows cyclic voltammograms of BMP-FLD fusion protein in the absence (1, thin line) and presence (2, thick line) of neomycin on glassy carbon electrode. Addition of carbon monoxide. Shifts the peak to higher potentials (3, dotted line). Potentials are reported versus saturated calomel electrode.

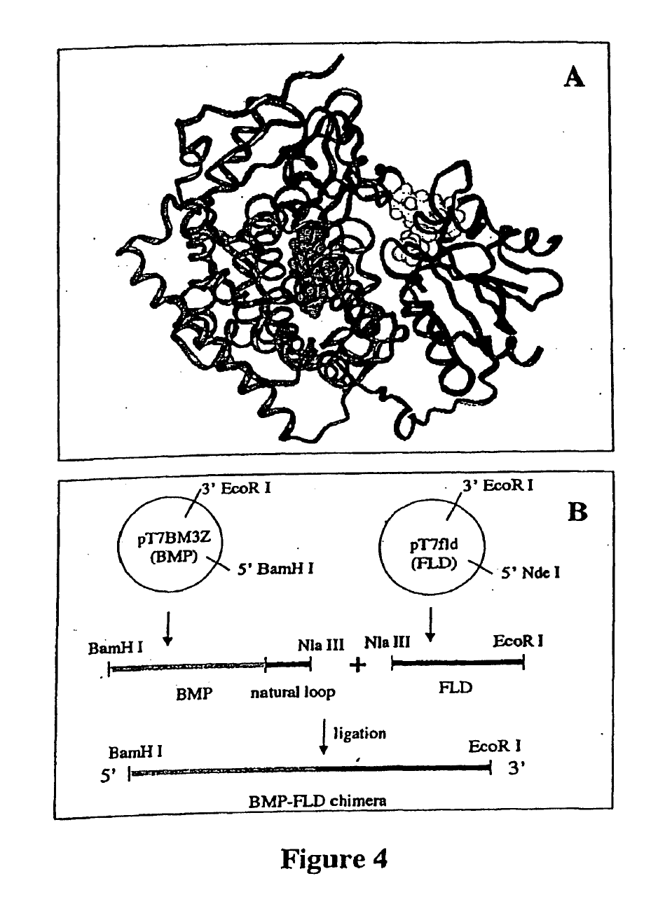

FIG. 4 shows the molecular biology approach to fuse the genes of BMP and FLD to generate the BMP-FLD chimera. The Nla III restriction sites were introduced by oligonucleotide directed mutagenesis.

The invention is illustrated further in the accompanying examples. The strategies adopted to tackle the three problems listed above by using the bacterial cytochrome P450 BM3 is shown in FIG. 1.

Cytochrome P450 BM3 is a soluble, catalytically self-sufficient fatty acid monoxygenase isolated from Bacillus megaterium (Narhi and Fulco, 1986 and 1987). It is particularly interesting in that it has a multi-domain structure, composed of three domains: one FAD, one FMN and one haem domain, fused on the same 119 kDa polypetidic chain of 1048 residues. Furthermore, despite its bacterial origin, P450 BM3 has been classified as a class II P450 enzyme, typical of microsomal eukaryotic P450s (Ravichandran et al., 1993): it shares 30% sequence identity with microsomal fatty acid w-hydroxylase, 35% sequence identity with microsomal NADPH:P450 reductase, and only 20% homology with other bacterial P450s (Ravichandran et al., 1993). These characteristics have suggested the use of P450 BM3 as a surrogate for mammalian P450s, and this has been recently substantiated when the structure of rabbit P450 2C5 was solved (Williams et al., 2000).

For these reasons, the haem domain of this enzyme is chosen in this work as an ideal candidate to be used for the molecular Lego approach to produce a P450 with the desired electrochemical properties. In particular, the efficient electron transfer of the P450 with the electrode surface, was tackled by choosing the haem domain (residues 1-470) of P450 BM3 (BMP) as a catalytic module to be fused by rational design with the flavodoxin from Desulfovibrio vulgaris to be used as an electron transfer module of well characterised electrochemical properties (FIG. 1A). In this design, the electron transfer module (flavodoxin) would facilitate the contact of the resulting P450 multi-domain construct with the electrode surface, allowing electrochemical accessibility of the buried P450 haem.

Direct electrochemistry of P450 enzymes with unmodified electrodes has in general proven very difficult due to the deeply buried haem cofactor and instability of the biological matrix upon interaction with the electrode surface. One solution to these problems is the modification of electrode surfaces. To date, most efforts have been focussed on characterisation of the electrochemistry of P450cam. This enzyme has been incorporated in lipid or polyelectrolyte film leading to well-defined redox behaviour from its haem Fe(II/III) (Zhang et al., 1997). More recently, the same enzyme was found to exhibit fast heterogeneous redox reaction on a glassy carbon electrode modified with sodium montmorillonite (Lei et al., 2000). Moreover, Hill and his colleagues (Kazlauskaite et al., 1996) using an edge-plane graphite electrode reported the first direct electrochemistry of P450cam in solution. The same group (Lo et al., 1999) demonstrated cyclic voltammograms on an edge-plane graphite electrode for various P450cam mutants. Nevertheless, to this date the electrochemistry of cytochrorne P450 BM3 has not been reported in the literature, despite its solubility and close relationship to the membrane-bound mammalian enzymes.

### Methods

Electron Transfer Measurements between P450BM3 Haem Domain (BMP) and Flavodoxin (FLD).

All absorbance measurements were carried out using a Hewlett-Packard 8452 diode array spectrophotometer. The wild type flavodoxin from D. vulgaris (FLD, 4.9 μM) in 5 mM potassium phosphate buffer pH 7.3 was photoreduced in the presence of 2.5 μM deazariboflavin (dRf) and 0.85 mM EDTA (sacrificial electron donor) to its semiquinone form (FLD_{sq}, equations [1] and [2] of the results section). Kinetic measurements were carried out following the reduction of the arachidonate bound BMP under carbon monoxide atmosphere, monitoring the absorbance at 450 nm in a Hi-Tech SF-61 stopped flow apparatus with a 1 cm path length cell, at 23° C. The typical arachidonate bound BMP concentration was 1 μM, and that of FLD was varied between 2-20 μM (equation [3] of the results section). Special care was taken to achieve anaerobic conditions by bubbling all solutions with argon.

Construction and Expression of the BMP-FLD Chimera.

The BMP-FLD fusion complex was constructed by introducing a Nla III site both at the 3′ end of the loop of P450 BM3 reductase gene in pT7BM3Z (Li et al., 1991) and 5′ end of the pT7FLD gene (Krey et al., 1988, Valetti et al., 1998). This was carried out by PCR using the mutagenic oligonucleotides sequence ID.1 (for BM3) and sequence ID.2 (for flavodoxin). The two genes were digested with Nla III endonuclease followed by a ligation step. The expression and purification of the wild type (wt) P450 BM3 and of the BMP-FLD chimera were carried out according to published protocols (Li et al., 1991, and Sadeghi et al., 2000a, respectively).

Sequence ID 1

CACAAGCAGCGGCATGTTATGAGCGTTTTC;
and

Sequence ID 2

AGGAAACAGCACATGCCTAAAGCTCTGATC.

Electron Transfer Measurements on the BMP-FLD Fusion Protein.

Steady-state photo-reduction of 4 μM BMP-FLD fusion protein was performed in 100 mM phosphate buffer pH 7 containing 5 μM deazariboflavin and 5 μM EDTA, under strict anaerobic conditions; photo-irradiation was carried out using a 100 W lamp. Laser flash photolysis was carried out as previously described (Hazzard et al., 1997). The BMP-FLD fusion protein (5 μM) was kept under strict anaerobic conditions in carbon monoxide saturated 100 mM phosphate buffer pH 7, containing 100 μM of deazariboflavin and 1 mM EDTA.

Electrochemical Experiments on the BMP-FLD Fusion Protein.

All electrochemical experiments were carried out with the Autolab PSTAT 10 controlled by the GPES software (Eco Chemie, Utrecht, NL). The staircase cyclic voltammetry was performed in a Hagen cell (Heering and Hagen, 1996) where the working electrode was glassy carbon disc with a platinum wire as the counter. The working electrode was activated and polished as previously described (Heering and Hagen, 1996). The reference electrode was Saturated Calomel with a potential of +246 mV versus the normal hydrogen electrode (NHE). All measurements were performed under strict anaerobic conditions with protein concentrations of 30 μM in 50 mM HEPES buffer pH 8.0, at 7° C.

Molecular Modelling.

All modelling studies and calculations were performed using the Biosym/MSI software installed on an SGI Indigo2 workstation, IRIX 6.2. Surface electrostatic potentials were calculated using the DelPhi 2.0 module under Insight II environment. DelPhi calculations were performed using a dielectric constant of 2.0 for the solute and 80 for the solvent with an ionic strength of 100 mM; solvent radius was set at 1.4 Å and ionic radius at 2.0 Å. The Poisson-Boltzmann algorithm was applied in its non-linear form with a limit of 2000 iteration and convergence of 0.00001 to a grid of resolutions ≦1.0 Å, centered around the protein. The minimal distance between the molecular surface and the grid boundary was 15.0 Å. Only formal charges were taken into account: the C- and N-terminus and the Glu, Asp, Arg and Lys side-chains were considered to be fully ionised, with the FMN phosphate and the haem iron (Fell) also included in the calculation. The solvent exposure was calculated using the Connolly algorithm (Connolly, 1983), with a probe of 1.4 Å radius. The Protein Data Bank (pdb) files used were the oxidised form of FLD (Watt et al., 1991), the P450terp (Hasemann et al., 1994), P450cam (Poulos et al., 1986), P450eryF (Cuppvickery and Poulos, 1995) and the haem domain of P450 BM3 (Ravichandran et al., 1993; Li and Poulos, 1997; Sevrioukova et al., 1999).

### EXAMPLE 1

The suitability of flavodoxin from D. vulgarlis (FLD) and the haem domain of cytochrome P450 BM3 from a B. megaterium (BMP) as electron transfer and catalytic modules to be used for the covalent assembly of a multi-domain construct was tested. The electron transfer (ET) between the separate proteins was studied by stopped-flow spectrophotometry. Flavodoxin (FLD_{q}) was reduced anaerobically under steady state conditions to its semiquinone form (FLD_{sq}) in one syringe of the stopped-flow apparatus by the semiquinone radical of deazaribofiavin (dRfH) produced by photo-irradiation in the presence of EDTA. The reaction scheme studied is summarised in the following equations (Sadeghi et al., 1999):

$$\begin{matrix} dRf \frac{hv}{EDTA} dRfH & [ 1 ] \\ dRfH + {FLD}^{-} dRf + {FLD}_{sq} & [ 2 ] \\ {FLD}_{sq} + {( BMP S )}_{OX} ⇄ [ {{FLD}_{sq} ( BMP \text{-} S )}_{OX} ] + {CO}^{-} {FLD}_{q} + {( BMP \text{-} S \text{-} CO )}_{red} & [ 3 ]\end{matrix}$$

Under pseudo-first order and saturating conditions, the ET process of the FLD_{sq}/(BMP-S)ₒₓ redox pairs showed an increase of the absorbance at 450 nm (FIG. 2A). This is consistent with the reduction of (BMP-S)ₒₓ, that promptly forms the carbon monoxide adduct responsible for the absorbance at 450 nm. The pseudo-first order rate constant (k_{obs}) was calculated by fitting the data points to a single exponential component. When the concentration of FLD_{sq} was varied between 2-20 μM, the k_{obs} was found to follow a saturating behaviour consistent with the formation of a complex between the two proteins. Fitting the data points of the k_{obs} versus the concentrations of FLD_{sq} to a hyperbolic function led to the limiting rate constant, kₗᵢₘ, of 43.77±2.18 s⁻¹ and to the apparent dissociation constant, Kₐₚₚ, of 1.23±0.32 μM at an ionic strength of 250 mM in 10 mM phosphate buffer, pH 7.3.

An important factor for achieving efficient ET is the formation of an ET competent complex between the redox pairs. The effect of the electrostatic forces in producing the complexes between BMP and FLD was studied by changing the ionic strength of the protein solutions. The resulting kₗᵢₘ values plotted against the square root of the ionic strength, I, showed the bell-shaped trend shown in FIG. 2B. This is usually due to hydrophobic as well as electrostatic interactions taking part in the formation of the complex (Sadeghi et al., 2000b). This was confirmed by the calculation of the surface potentials of the two proteins shown in FIG. 3.

The availability of the 3D structures of chosen protein modules allows the use of computational methods for generating a 3D model of the possible complexes. The structure of such models is important in this work for the rational design of the covalently linked assembly described here.

A model for the FLD/BMP complex was generated by super-imposition of the 3D structure of FLD on that of the truncated P450 BM3 (Sevrioukova et al., 1999). The distance between the redox centers in this complex is 18 Å, which is comparable with that found in the structure of the truncated P450 BM3 (Sevrioukova et al., 1999). However, an alternative model is also possible, where the FMN region of FLD is docked in the positively charged depression on the proximal BMP surface, around the haem ligand cysteine 400. This model brings the two cofactors at a closer distance of <12 Å. The two possible models may reflect the presence of dynamic events accompanying the formation and reorganisation of the ET competent complex that has also been postulated for the natural P450-reductase complex (Williams et al., 2000).

The model of the ET competent complex described above was used to generate a covalently linked complex of BMP-FLD. This was achieved by linking a flexible connecting loop introduced by gene fusion as shown in FIG. 4B. This method offers the advantage of keeping the two redox domains in a dynamic form. The fusion of the BMP-FLD system was carried out at DNA level by linking the BMP gene (residues 1-470) with that of FLD (residues 1-148) through the natural loop of the reductase domain of P450 BM3 (residues 471-479). The gene fusion was achieved by ligation of the relevant DNA sequences with engineered Nla III restriction sites.

The fusion gene was heterologously expressed in a single polypeptide chain in E. coli BL21 (DE3) C1. The absorption spectra of the purified chimeric protein indicated the incorporation of 1:1 haem and FMN. Moreover, the reduced protein was able not only to form the carbon monoxide adduct with the characteristic absorbance at 450 nm, but also to bind substrate (arachidonate) displaying the expected low- to high-spin transition from 419 nm to 397 nm, indicating that this covalent complex is indeed a functional P450. The integrity of the secondary structure of the BMP-FLD fusion protein was confirmed by CD spectroscopy (data not shown); with a ˜2% increase in the a-helix content when compared to the BMP, probably due to the addition of the engineered loop. The spectroscopic data show that the fusion protein is indeed expressed as a soluble, folded and functional protein (Sadeghi et al.; 2000a).

The presence of intra-molecular ET in the BMP-FLD fusion protein, from the domain containing the FMN to the domain containing the haem, in the presence of substrate, was studied under steady-state conditions. The flavin domain was photoreduced by deazariboflavin in the presence of EDTA under anaerobic conditions. The subsequent ET from the flavin domain to the haem was followed by the shift of the haem absorbance from 397 nm to 450 nm in carbon monoxide saturated atmosphere. The kinetics of the intra-molecular ET within the BMP-FLD fusion protein was studied by transient absorption spectroscopy. In the experimental set up, the FMN-to-haem ET was followed by the decrease in absorbance at 580 nm of the FLD_{sq}. The ET rate measured was found to be 370 s⁻¹. This value is comparable to that measured for the intra-protein ET from FMN to haem domain of truncated P450 BM3 (250 s⁻¹) in which the FAD domain was removed (Hazzard et al., 1997). These results are extremely encouraging because they demonstrate the functionality of the BMP-FLD fusion protein to be equivalent to the physiological protein.

Preliminary electrochemical experiments of the BMP-FLD fusion protein were carried out using a glassy carbon electrode. The cyclic voltammograms (cv) of both the BMP-FLD fusion-protein and BMP are shown in FIG. 3. While no current was observed for P450 BM3 enzyme on the bare glassy carbon electrode, the BMP-FLD shows measurable redox activities (thin line, FIG. 3). In particular, the BMP-FLD fusion protein interacts better with the electrode as measured by the larger current (thick line, FIG. 3) observed in the presence of neomycin, a positively charged aminoglycoside which is believed to overcome the electrostatic repulsion between the negatively charged FLD and the negatively charged electrode surface (Heering and Hagen, 1996). The enhancement of the current obtained in the presence of neomycin observed for BMP-FLD supports the hypothesis that FLD assists the electrochemical contact between the electrode and BMP. Efforts are currently made to achieve full electrochemical reversibility, as the lower current observed in the oxidative scan is consistent with oxygen leakage in the electrochemical cell. The results are consistent with the electrochemical response of the P450 haem, as supported by the shift at higher potentials in the cv obtained after addition of carbon monoxide (dotted line, FIG. 3).

The data prove that indeed non-physiological electron transfer between the BMP catalytic module and the FLD electron transfer module and between FLD and an electrode are possible, and the covalently linked multi-domain construct BMP-FLD exhibits improved electrochemical properties compared to wild-type BMP.

### REFERENCES

Bagby, S. B., Barker, P. D., Hill; H. A. O., Sanghera, G. S., Dunbar, B., Ashby, G. A., Eady, R. R., and Thorneley, R. N. F. (1992) Direct electrochemistry of two genetically distinct flavodoxins isolated from Aztobacter chroococcum grown under nitrogen fixing conditions. Biochem. J. 277, 313-319.
Connolly, M. L., (1983) Solvent-accessible surfaces of proteins and nucleic acids. Science, 221, 709-713.
Cuppvickery, J. R. and Poulos, T. (1995) Structure of P450eryF involved in erythromycin biosynthesis. Nature Struct. Biol. 2(2), 144-153.
Guengerich, F. P. (1999) Cytochrome P450: regulation and role in drug metabolism, Annu. Rev. Pharmacol. Toxicol. 39, 1-17.
Hasemann, C. A., Ravichandran, K. G., Peterson, J. A., and Deisenohofer, J. (1994) Crystal structure and refinement of cytochrome P450terp at 2.3 A resolution. J Mol Biol 236, 1169-1185.
Hazzard, J. T., Govindaraj, S., Poulos, T. L. and Tollin, G. (1997) Electron transfer between the FMN and haem domains of cytochrome P450 BM3. J. Biol. Chem., 272, 7922-7926.
Heering, H. A., and Hagen, W. R. (1996) Complex electrochemistry of flavodoxin at carbon-based electrodes: results from a combination of direct electron transfer, flaving-mediated electron transfer and comproportionation. J. Electroanal. Chem., 404-249-260.
Kazlauskaite, J., Westlake, A. C. G., Wong, L.-L., Hill, H. A. O. (1996) Direct electrochemistry of cytochrome P450cam. Chem. Commun. 18, 2189-2190.
Krey, G. D., Vanin, E. F. and Swenson, R. P. (1988) Cloning, nucleotide sequence and expression of the flavodoxin gene from D. vulgaris (Hildenborough). J. Biol. Chem., 263, 15436-15443.
Lei, C., Wollenberger, U., Jung, C., Scheller, F. W. (2000). Clay-bridged electron transfer between cytochrome P450ₐₘ and electrode. Biochem. Biophys. Res. Commun. 268, 740-744.
Li, H. and Poulos, T. L., (1997) The structure of the cytochrome P450 BM-3 haem domain complexed with the fatty acid substrate, plamitoleic acid. Nature Str. Biol., 4, 140-146.
Li, H., Darwish, K. and Poulos, T. (1991) Characterisation of recombinant B. megaterium cytochrome P450 BM3 and its functional domains. J. Biol. Chem., 266, 11909-11914.
Lo, K. K. W., Wong, L.-L., Hill, H. A. O. (1999). Surface-modified mutants of cytochrome P450(cam): enzymatic properties and electrochemistry. FEBS Lett. 451, 342-346.
Narhi, L. O., and Fulco, A. J. (1986) Characterization of a catalytically self-sufficient 119,000-Dalton cytochrome P-450 monooxygenase induced by barbiturates in Bacillus megaterium. J. Biol. Chem., 261(16), 7160-7169.
Narhi, L. O., and Fulco, A. J. (1987) Identification and characterization of two functional domains in cytochrome P-450 BM3, a catalytically self-sufficient monooxygenase induced by barbiturates in Bacillus megaterium. J. Biol. Chem., 262 (14), 6683-6690.
Poulos, T. L. (1995) Cytochrome P450. Curr. Opin. Struct. Biol., 5, 767-774.
Poulos, T. L., Finzel, B. C., Howard, A. J. (1986) Crystal structure of substrate-free Pseudomonas putida cytochrome P450. J Am Chem Soc 25, 5314-5322.
Ravichandran, K. G., Boddupalli, Hasemann; C. A., Peterson, J. A., and Deisenhofer, J. (1993) Crystal structure of hemoprotein domain of P450BM-3, a prototype for microsomal P450s. Science, 261, 731-736.
Sadeghi, S. J., Meharenna, Y. T., and Gilardi, G. (1999) Flavodoxin as a module for transferring electrons to different c-type and P450 cytochromes in artificial redox chains. In: Ghisla, S., Kroneck, P., Macheroux, P., Sund, H. (Eds.), Flavins and flavoproteins. Agency for Scient. Publ., Berlin, pp. 163-166.
Sadeghi, S. J., Meharenna, Y. T., Fantuzzi, A., Valetti, F. and Gilardi, G. (2000a) Engineering artificial redox chains by molecular Lego, Faraday Discuss., 116, 135-153.
Sadeghi, S., Valetti, F., Cunha, C A., Romao, M. J., Soares, C. M. and Gilardi, G. (2000b) Ionic strength dependence of the non-physiological electron transfer between flavodoxin and cytochrome c553 from D. vulgaris, J. Biol. Inorg. Chem. 5 (6); 730-737.
Sadeghi, S. J., Tsotsou, G. E., Fairhead, M., Meharenna, Y. T. and Gilardi, G. (2001) Rational design of P450 enzymes for biotechnology, In: Focus on Biotechnology. Physics and Chemistry Basis of Biotechnology. De Cuyper, M. and Butte, J. (Eds), Kluwer Academic Publisher, in press.
Sevrioukova, I. F., Hazzard, J. T., Tollin, G., and Poulos, T. L. (1999). The FMN to Heme Electron Transfer in Cytochronme P450BM-3. J. Biol. Chem., 274(51), 36097-36106.
Valetti, F., Sadeghi, S. J., Meharenna, Y., Gilardi, G. (1998) Engineering multidomain redox proteins containing flavodoxin as bio-transformer: preparatory studies by rational design. Biosens. Bioelectron. 13, 675-685.
Watt, W., Tulinsky, A., Swenson, R. P., and Watenpaugh, K. D. (1991) Comparison of the crystal structures of a flavodoxin in its three oxidation states at cryogenic temperatures. J. Mol. Biol., 218, 195-208.
Williams, P. A., Cosme, J., Sridhar, V., Johnson, E. F., and McRee, D. E. (2000) Mammalian microsomal cytochrome P450 monooxygenase: Structural adaptations for membrane binding and functional diversity. Mol. Cell., 5, 121-131.
Zhang, Z., Nassar, A-E. F., Lu, Z. Q, Schenkman, J. B., Rusting, J. F. (1997) Direct electron injection from electrodes to cytochrome P450(cam) in biomembrane-like films. J. Chem. Soc.-Faraday Trans. 93(9), 1769-1774.

All of the aforementioned references are herein incorporated by reference in their entirety, essential and non-essential matter included, under 37 C.F.R. § 1.57.

What is claimed is:

1. A method in which a chimeric protein comprising a redox catalytic domain derived from a first source and an electron transfer domain derived from a second source different to the first source is contacted with a substrate for the catalytic domain, and with an electrode, whereby the substrate is acted on by the catalytic domain, to form a product, and electrons are transferred between the electrode and the electron transfer domain and directly between the electron transfer domain and the catalytic domain.

2. A method according to claim 1, wherein the redox catalytic domain is a haem-containing domain.

3. A method according to claim 2, wherein the haem-containing domain is a monooxygenase domain.

4. A method according to claim 1, wherein the electron transfer domain is a haem reductase domain and the electrode is a cathode.

5. A method according to claim 1, wherein the electron transfer domain is a flavoprotein.

6. A method according to claim 5, wherein the flavoprotein is flavodoxin from D. vulgaris or an active electron-transferring mutant form thereof.

7. A method according to claim 1, wherein electrons are directly transferred from the electrode to the electron transfer domain.

8. A method according to claim 1, wherein the chimeric protein additionally comprises a docking sequence having a docking site for the electron transfer domain.

9. A method according to claim 8, wherein the electron transfer docking sequence is derived from the same source as the redox catalytic domain.

10. A method according to claim 3, wherein the source of the redox domain is a cytochrome P450.

11. A method according to claim 10, wherein the redox catalytic domain is derived from a bacterial cytochrome P450 enzyme.

12. A method according to claim 11, wherein the enzyme is BM3 of Bacillus megaterium.

13. A method according to claim 1, wherein the flow of electrons to or from the electrode is measured using a current or voltage detector.

14. A method according to claim 13, wherein the substrate is an analyte of interest and measurement of the flow electrons is used to detect the presence or amount of substrate.

15. A method according to claim 1, wherein the electrons are driven from the electrode, the substrate is consumed and the product is separated from the chimeric protein and recovered.

16. A method according to claim 15, wherein the chimeric protein is immobilised on the electrode, the substrate is initially present in solution in contact with the immobilised enzyme and the product is recovered from solution.


SEQUENCE LISTING (2 sequences)

SEQ ID NO: 1 - DNA, 30 bp, Artificial Sequence
  Description of Artificial Sequence Synthetic oligonucleotide
cacaagcagcggcatgttatgagcgttttc

SEQ ID NO: 2 - DNA, 30 bp, Artificial Sequence
  Description of Artificial Sequence Synthetic oligonucleotide
aggaaacagcacatgcctaaagctctgatc